(19) United States
(12) Patent Application Publication
(10) Pub. No.: US 2025/0073295 A1
(43) Pub. Date: Mar. 6, 2025

(54) ANTI-OBESITY COMPOSITION CONTAINING HEMP SEED EXTRACT AS ACTIVE INGREDIENT

(71) Applicant: Andong National University Industry-Academic Cooperation Foundation, Andong-si (KR)

(72) Inventors: Jee Hye SUNG, Daegu (KR); Ye Ji KWON, Andong-si (KR)

(73) Assignee: Andong National University Industry-Academic Cooperation Foundation, Andong-si (KR)

(21) Appl. No.: 18/594,709

(22) Filed: Mar. 4, 2024

(30) Foreign Application Priority Data
Sep. 6, 2023 (KR) 10-2023-0118279

(51) Int. Cl. A61K 36/185; A61K 9/00; A61P 3/04

(52) U.S. Cl. CPC A61K 36/3482; A61K 9/0056; A61P 3/04; A61K 2236/35; A61K 2236/37

(57) ABSTRACT

Proposed is an anti-obesity composition containing a hemp seed extract as an active ingredient. Specifically, food and pharmaceutical compositions for preventing or reducing obesity, each of which contains a hemp seed extract as an active ingredient, are proposed. Additionally, proposed is a method of preparing a hemp seed unsaponifiable extract having an anti-obesity activity. The method includes (1) adding a hemp seed, pyrogallol, and potassium hydroxide (KOH) to a reaction vessel and filling the reaction vessel with nitrogen, (2) causing a reaction in a water bath at a temperature in a range of 70° C. to 80° C. for 40 to 60 minutes and then cooling the resulting product, and (3) adding sodium chloride (NaCl) and an extraction solvent in which dibutylhydroxytoluene (BHT) hexane and ethyl acetate are mixed to the cooled product and shaking the resulting mixture to obtain a supernatant.

### CROSS REFERENCE TO RELATED APPLICATION

The present application claims priority to Korean Patent Application No. 10-2023-0118279, filed Sep. 6, 2023, the entire contents of which is incorporated herein for all purposes by this reference.

### STATEMENT REGARDING GOVERNMENT SPONSORED RESEARCH

This invention was supported by the Basic Science Research Program through the National Research Foundation of Korea funded by the Ministry Education, Science and Technology, Republic of Korea [project number 2018R1A6A1A03024862]

### BACKGROUND OF THE DISCLOSURE

### 1. Field of the Disclosure

The present disclosure relates to an anti-obesity composition containing a hemp seed extract as an active ingredient.

### 2. Description of the Related Art

Obesity is a medical condition characterized by excessive fat accumulation caused by imbalanced energy intake and consumption, leading to increases in the number and size of fat cells. Energy in the body is stored in a triglyceride form in the fat cells. Once energy sources are depleted, such stored energy is broken down into free fatty acids and glycerol and thus used as an energy source. However, excessive energy intake promotes fat cell differentiation and increases the amount of fat stored in the body, which is a direct cause of obesity.

Obesity not only changes body shape due to fat accumulation in the viscera and abdomen but also acts as a risk factor that increases the incidence rate of various diseases. Excessive accumulation of visceral fat causes problems in sugar metabolism in the body and symptoms, including abnormal hormone secretion and abnormal cytokine secretion. While obesity causes increases in triglyceride and low-density lipoprotein (LDL) cholesterol levels, decreases in high-density lipoprotein (HDL) cholesterol levels cause abnormalities in lipid metabolism in the body, decrease the number of insulin receptors present in the tissue, and reduce insulin sensitivity, thus inhibiting the transport of glucose into cells while causing hyperglycemia and diabetes. Additionally, obesity is known to be closely related to the occurrence of metabolic diseases, including hyperlipidemia, cardiovascular diseases, cancer, respiratory disorders, stroke, and osteoarthritis.

Furthermore, due to the issues regarding supply and excessive intake of food that promote obesity, such as fast food, obesity is being regarded as a social problem in Korea and around the world. Thus, a great deal of research is in progress on developing anti-obesity drugs capable of preventing, reducing, and treating obesity.

However, existing anti-obesity drugs such as orlistat and sibutramine are known to cause serious side effects, including vomiting, constipation, gastrointestinal disorders, and cardiovascular diseases. Thus, there is a need to develop even more effective and safer anti-obesity drugs.

On the other hand, hemp seed is the seed of hemp, which is a type of Cannabis Sativa and whose scientific name is Cannabis sativa L., and contains gamma-linolenic acid (GLA), which is known to be effective in preventing and treating skin aging, neurodermatitis, cardiovascular diseases, rheumatoid arthritis, and the like. Yet, no research has been conducted on the anti-obesity activity thereof.

### DOCUMENT OF RELATED ART

### Patent Document

(Patent Document 0001) Korean Patent No. 10-2305606

### SUMMARY OF THE INVENTION

Hence, the inventors of the present disclosure demonstrated that a hemp seed extract, specifically, unsaponifiable hemp seed extract, had an excellent anti-obesity activity, thereby completing the present disclosure.

Therefore, the present disclosure aims to provide a food composition for preventing or reducing obesity, the food composition containing a hemp seed extract as an active ingredient.

Additionally, the present disclosure aims to provide a pharmaceutical composition for preventing or treating obesity, the pharmaceutical composition containing a hemp seed extract as an active ingredient.

Furthermore, the present disclosure aims to provide a method of preparing an unsaponifiable hemp seed extract having an anti-obesity activity.

To achieve the objectives of the present disclosure as described above, the present disclosure provides a food composition for preventing or reducing obesity, the food composition containing a hemp seed extract as an active ingredient.

In one example of the present disclosure, the hemp seed extract may be an unsaponifiable hemp seed extract.

In one example of the present disclosure, the hemp seed extract may have the following activities: inhibiting lipid droplet formation and adipocyte differentiation and reducing adipocyte size, when inducing preadipocytes to be differentiated into adipocytes; and reducing total triglyceride, total cholesterol, and low-density lipoprotein (LDL) cholesterol levels.

Additionally, the present disclosure provides a pharmaceutical composition for preventing or treating obesity, which contains a hemp seed extract as an active ingredient.

In one example of the present disclosure, the hemp seed extract may be an unsaponifiable hemp seed extract.

In one example of the present disclosure, the hemp seed extract may have the following activities: inhibiting lipid droplet formation and adipocyte differentiation and reducing adipocyte size, when inducing preadipocytes to be differentiated into adipocytes; and reducing total triglyceride, total cholesterol, and low-density lipoprotein (LDL) cholesterol levels.

Additionally, the present disclosure provides a method of preparing an unsaponifiable hemp seed extract having an anti-obesity activity. The method includes (1) adding a hemp seed, pyrogallol, and potassium hydroxide (KOH) to a reaction vessel and filling the reaction vessel with nitrogen, (2) causing a reaction in a water bath at a temperature in a range of 70° C. to 80° C. for 40 to 60 minutes and then cooling the resulting product, and (3) adding sodium chloride (NaCl) and an extraction solvent in which dibutylhydroxytoluene (BHT) hexane and ethyl acetate are mixed to the cooled product and shaking the resulting mixture to obtain a supernatant.

A hemp seed extract, according to the present disclosure, has the following activities: inhibiting lipid droplet formation and adipocyte differentiation and reducing adipocyte size, when inducing preadipocytes to be differentiated into adipocytes; and reducing total triglyceride, total cholesterol, and low-density lipoprotein (LDL) cholesterol levels. Thus, the hemp seed extract can be effectively usable in the production of health functional foods and pharmaceutical drugs for preventing, reducing, and treating obesity.

### BRIEF DESCRIPTION OF THE DRAWINGS

FIGS. 1A and 1B show results of analyzing lipogenesis inhibitory effects by treatment with an unsaponifiable hemp seed extract of the present disclosure when inducing preadipocytes to be differentiated into adipocytes, where FIG. 1A is a graph showing the degree of total lipid accumulation, and FIG. 1B shows images of fat globules observed with a microscope after being stained;

FIGS. 2A and 2B show results of measuring changes in the body weight of mice, in which obesity is induced by a high-fat diet, by treatment with an unsaponifiable hemp seed extract of the present disclosure, and images of the mice;

FIG. 3 shows observation images of the size of the liver, epididymal white adipose tissue (eWAT), and mesenteric white adipose tissue (mWAT) isolated from mice in a normal diet group, a high-fat diet group (obese group), and high-fat diet groups treated with an unsaponifiable hemp seed extract of the present disclosure; and

FIGS. 4A and 4B show pathological observation images of the liver and epididymal white adipose tissue (eWAT) isolated from mice in a normal diet group, a high-fat diet group (obese group), and high-fat diet groups treated with an unsaponifiable hemp seed extract of the present disclosure.

### DESCRIPTION OF THE PREFERRED EMBODIMENTS

The present disclosure is characterized by providing a food composition for preventing or reducing obesity, the food composition containing a hemp seed extract as an active ingredient.

While conducting research to discover materials having anti-obesity activities from natural substances, the inventors of the present disclosure confirmed from experiments that a hemp seed extract had excellent anti-obesity activities.

In one example of the present disclosure, a hemp seed extract, specifically, an unsaponifiable hemp seed extract, was treated when inducing preadipocytes to be differentiated into adipocytes. As a result, it was shown that the hemp seed extract had activities capable of inhibiting lipid droplet formation and adipocyte differentiation and reducing adipocyte size.

Through these results, the inventors of the present disclosure were able to predict that the hemp seed extract of the present disclosure was effectively usable as an anti-obesity material. To prove this, mice in which obesity was induced by a high-fat diet were treated with the hemp seed extract of the present disclosure, and then whether there were anti-obesity effects was analyzed.

As a result, in the case of mice in groups treated with the hemp seed extract of the present disclosure, body weight gain was inhibited compared to that in the case of obese mice, thus shown to be effective in body weight loss. Additionally, the weight of the liver and adipose tissue was also effectively reduced.

Furthermore, the obesity-induced mice were administered the hemp seed extract of the present disclosure. As a result, it was shown that plasma lipid and cholesterol concentrations were effectively reduced compared to those in the case of obese mice. Furthermore, all the total triglyceride, total cholesterol, and low-density lipoprotein cholesterol contents were reduced.

Additionally, it was confirmed that liver toxicity was not observed in all experimental mice treated with the hemp seed extract of the present disclosure, and cytotoxicity was not found even in experiments performed on preadipocytes, showing that the hemp seed extract of the present disclosure was not harmful to the body.

Through these results, the inventors of the present disclosure found that the hemp seed extract, according to the present disclosure, was effectively usable to prevent, reduce, and treat obesity.

Therefore, the present disclosure may provide the food composition for preventing or reducing obesity, the food composition containing the hemp seed extract as the active ingredient.

In the present disclosure, the hemp seed extract refers to a concentrated formulation obtained by squeezing a natural material to prepare a suitable leachate and evaporating the resulting leachate, but is not limited thereto. The hemp seed extract may be an extracted liquid obtained through an extraction process, a diluted or concentrated liquid of the extracted liquid, a dried product obtained by drying the extracted liquid, or a crudely purified product or a purified product thereof. The hemp seed extract may be prepared using typical extraction, isolation, and purification methods known in the art.

Preferably, the hemp seed extract of the present disclosure is an unsaponifiable hemp seed extract.

The “unsaponifiable extract”, that is, “unsaponifiable matter”, includes all fatty components that are poorly soluble or insoluble in water after saponification in a strongly alkaline medium while being soluble in organic solvents, such as ethyl ether, aromatic hydrocarbons, and chlorinated solvents. The unsaponifiable hemp seed extract of the present disclosure refers to an extract containing unsaponifiable components from hemp seeds as alkali.

As used herein, the term “food composition” may refer to food that acts beneficially on one or more functions of an organism, regardless of nutrients provided to a subject consuming the same, and thus improves a physical condition. Accordingly, the food composition is usable to prevent, reduce, or treat diseases or disease-causing factors.

Therefore, the term “food composition” used herein may be a synonym for functional food, food for specific nutritional purposes, or medical food.

In the food composition of the present disclosure, typical pharmaceutically acceptable carriers or excipients may be further contained in addition to the active ingredient of the present disclosure. Additionally, the food composition may be prepared through formulation with combinations of various pharmaceutical additives commonly used, such as binders, disintegrants, coating agents, lubricants, and the like, for preparation.

The food composition of the present disclosure may contain a variety of flavoring agents, natural carbohydrates, or the like as additional components, like a normal food composition, in addition to the active ingredient of the present disclosure.

Examples of the natural carbohydrates mentioned above may include monosaccharides, such as glucose, fructose, and the like, disaccharides, such as maltose, sucrose, and the like, typical sugars including polysaccharides, such as dextrin, cyclodextrin, and the like, and sugar alcohols, such as xylitol, sorbitol, erythritol, and the like. As the flavoring agents mentioned above, natural flavoring agents (thaumatin), stevia extracts (for example, rebaudioside A, glycyrrhizin, and the like), and synthetic flavoring agents (saccharin, aspartame, and the like) may be beneficially used.

Additionally, the food composition of the present disclosure is preferably formulated as a food composition by containing one or more sitologically or pharmaceutically acceptable carriers in addition to the active ingredient of the present disclosure.

The food composition may be formulated in the form of a tablet, a capsule, a powder, a granule, a liquid, a pill, a solution, a syrup, a juice, a suspension, an emulsion, a drop, or the like. For example, to be formulated in a tablet or capsule form, the active ingredient may be combined with an oral, non-toxic, and pharmaceutically acceptable inert carrier, such as ethanol, glycerol, water, or the like. Additionally, a suitable binder, a lubricant, a disintegrant, and a coloring agent may also be contained in the form of a mixture, if desired or necessary. Examples of the suitable binders may include starch, gelatin, a natural sugar such as glucose or beta-lactose, natural and synthetic gums such as a corn sweetener, acacia, tragacanth, or sodium oleate, sodium stearate, magnesium stearate, sodium benzoate, sodium acetate, sodium chloride, and the like, but are not limited thereto. Examples of the disintegrants may include starch, methylcellulose, agar, bentonite, xanthan gum, and the like, but are not limited thereto. As the acceptable pharmaceutical carriers for compositions formulated as liquid solutions, those suitable for sterilization and biocompatibility, including a saline solution, sterile water, Ringer's solution, a buffered saline solution, ab albumin injection solution, a dextrose solution, a maltodextrin solution, glycerol, ethanol, and a mixture of one or more thereof, may be used. Additionally, other common additives such as antioxidants, buffers, and bacteriostats may be added as needed. Furthermore, diluents, dispersants, surfactants, binders, and lubricants may be further added to formulate an injectable formulation, such as an aqueous solution, a suspension, and an emulsion, a pill, a capsule, a granule, or a tablet.

The food composition of the present disclosure, formulated in such a manner as described above, may be used as a functional food, or may be added to all kinds of food.

Examples of food to which the composition of the present disclosure may be added may include beverages, meat, chocolate, foodstuffs, confectionery, pizza, ramen, other noodles, gum, candy, ice cream, alcoholic beverages, vitamin complexes, health supplements, and the like.

In addition to the active ingredient of the present disclosure, the food composition may contain various nutrients, vitamins, minerals (electrolytes), flavoring agents such as synthetic and natural flavors, colorants, extenders (cheese, chocolate, and the like), pectic acid and salts thereof, alginic acid and salts thereof, organic acids, protective colloidal thickeners, pH adjusters, stabilizers, preservatives, glycerin, alcohols, carbonating agents used in carbonated drinks, and the like. Additionally, the food composition of the present disclosure may contain pulp to produce natural fruit juices, fruit juice drinks, and vegetable beverages.

Additionally, the present disclosure may provide an anti-obesity pharmaceutical composition containing the hemp seed extract as an active ingredient. Specifically, provided is a pharmaceutical composition for preventing or treating obesity, the pharmaceutical composition containing the hemp seed extract as the active ingredient.

As used herein, the term “anti-obesity” may refer to the prevention, treatment, or reduction of obesity. Accordingly, the term “anti-obesity composition” used herein may refer to the pharmaceutical composition for preventing or treating obesity.

As used herein, the term “prevent”, “preventing”, or “prevention” may refer to all actions that inhibit or delay the onset of obesity with the administration of the pharmaceutical composition according to the present disclosure. Additionally, the term “treat”, “treating”, or “treatment” may refer to all actions that reduce or beneficially change the symptoms of an individual with a suspected or developed obesity.

In the present disclosure, the pharmaceutical composition may contain a pharmaceutically acceptable carrier. The term “pharmaceutically acceptable” used herein may mean not to substantially stimulate the organism and not to inhibit the biological activity and characteristics of an active substance being administered.

As used herein, the term “treat”, “treating”, or “treatment” may refer to all actions that reduce or beneficially change the symptoms of obesity with the administration of the composition of the present disclosure.

As used herein, the term “reduce”, “reducing”, or “reduction” may mean all actions that lead to a reduction in at least one parameter associated with the condition being treated, such as the severity of symptoms.

As used herein, the term “administer”, “administering”, or “administration” may mean to provide an individual with the given composition of the present disclosure by a suitable method. In this case, the individual may refer to all animals, such as humans, monkeys, dogs, goats, pigs, or rats, whose symptoms of obesity may be reduced with the administration of the composition of the present disclosure.

As used herein, the term “pharmaceutically effective amount” may mean an amount sufficient to treat a disease with a reasonable benefit or risk ratio applicable to medical treatment, and may be determined on the basis of factors, including the type of disease, severity, drug activity, sensitivity to drugs, administration time, administration route, excretion rate, treatment duration, and drugs used simultaneously, and other factors well known in the field of medicine. For example, despite not being constant, the effective amount typically administered may be in a range of 0.001 to 100 mg/kg and is preferably in the range of 0.01 to 10 mg/kg, once or several times a day. The dosage does not limit the scope of the present disclosure in any way.

Additionally, the pharmaceutical composition of the present disclosure may be prepared using pharmaceutically suitable and physiologically acceptable supplements in addition to the hemp seed extract of the present disclosure. As the supplements, excipients, binders, disintegrants, sweeteners, coating agents, leavening agents, lubricants, flavoring agents, or the like may be used.

For administration, the pharmaceutical composition may be preferably formulated as a pharmaceutical composition by further containing one or more pharmaceutically acceptable carriers in addition to the active ingredient of the present disclosure.

The pharmaceutical composition may be formulated in the form of a granule, a powder, a tablet, a coated tablet, a capsule, a suppository, a solution, a syrup, a juice, a suspension, an emulsion, a drop, or an injectable solution. For example, to be formulated in a tablet or capsule form, the active ingredient may be combined with an oral, non-toxic, and pharmaceutically acceptable inert carrier, such as ethanol, glycerol, water, or the like. Additionally, a suitable binder, a lubricant, a disintegrant, and a coloring agent may also be contained in the form of a mixture, if desired or necessary. Examples of the suitable binders may include starch, gelatin, natural sugars such as glucose or beta-lactose, natural and synthetic gums such as a corn sweetener, acacia, tragacanth, or sodium oleate, sodium stearate, magnesium stearate, sodium benzoate, sodium acetate, sodium chloride, and the like, but are not limited thereto. Examples of the disintegrants may include starch, methylcellulose, agar, bentonite, xanthan gum, and the like, but are not limited thereto. As the acceptable pharmaceutical carriers for compositions formulated as liquid solutions, those suitable for sterilization and biocompatibility, including a saline solution, sterile water, Ringer's solution, a buffered saline solution, ab albumin injection solution, a dextrose solution, a maltodextrin solution, glycerol, ethanol, and a mixture of one or more thereof, may be used. Additionally, other common additives such as antioxidants, buffers, and bacteriostats may be added as needed. Furthermore, diluents, dispersants, surfactants, binders, and lubricants may be further added to formulate an injectable formulation, such as an aqueous solution, a suspension, and an emulsion, a pill, a capsule, a granule, or a tablet. Moreover, the composition is preferably formulated according to each disease or type of component, using the method disclosed in Remington's Pharmaceutical Science, Mack Publishing Company, Easton PA as an appropriate method in the field to which the present disclosure pertains.

Additionally, the present disclosure provides a method of preparing an unsaponifiable hemp seed extract having an anti-obesity activity. The method includes (1) adding a hemp seed, pyrogallol, and potassium hydroxide (KOH) to a reaction vessel and filling the reaction vessel with nitrogen, (2) causing a reaction in a water bath at a temperature in a range of 70° C. to 80° C. for 40 to 60 minutes and then cooling the resulting product, and (3) adding sodium chloride (NaCl) and an extraction solvent in which dibutylhydroxytoluene (BHT) hexane and ethyl acetate are mixed to the cooled product and shaking the resulting mixture to obtain a supernatant.

That is, in the method according to the present disclosure, a solution of pyrogallol and potassium hydroxide is first added to the hemp seed, followed by inducing saponification at a temperature of 70° C. to 80° C. for 40 to 60 minutes, and then the resulting product is cooled. Next, sodium chloride and the extraction solvent in which dibutylhydroxytoluene (BHT) hexane and ethyl acetate are mixed are added to the cooled product, and the resulting mixture is shaken to obtain the supernatant, which is the unsaponifiable hemp seed extract.

Preferably, the mixed extraction solvent used is an extraction solvent in which dibutylhydroxytoluene (BHT) hexane and ethyl acetate are mixed in a volume ratio of 85:15.

Additionally, after the completion of shaking, the supernatant is obtained through layer separation, further concentrated under reduced pressure to remove the solvent, and then subjected to a freeze-drying process, thereby obtaining the unsaponifiable hemp seed extract according to the present disclosure.

Additionally, the inventors of the present disclosure performed component analysis on the unsaponifiable hemp seed extract prepared by the method of the present disclosure. As a result, it was confirmed that there were large amounts of vitamin E, policosanol, phytosterol, and carotenoid components contained.

Hereinafter, the present disclosure will be described in more detail through examples. These embodiments are disclosed only for illustrative purposes, and the scope of the present disclosure is not limited thereto.

### Example 1

### Preparation of Unsaponifiable Hemp Seed Extract

An unsaponifiable hemp seed extract was obtained using a saponification extraction method. Specifically, for saponification, about 4.0 g of a hemp seed sample was added to a saponification reaction vessel, and 20 mL of 6% pyrogallol and 8 mL of 6% KOH were added, followed by filling the reaction vessel with nitrogen. A reaction occurred in a water bath at a temperature of 75° C. for 50 minutes, and then the resulting product was cooled in an ice box. After adding 30 mL of 2% NaCl and 20 mL of an extraction solvent (0.01% BHT hexane:ethyl acetate=85:15) to the cooled product and shaking the resulting product, the supernatant was extracted through layer separation, concentrated under reduced pressure to remove the solvent, and then subjected to freeze drying, thereby obtaining the unsaponifiable hemp seed extract of the present disclosure. Additionally, the obtained extract was stored at a temperature of −20° C. and used as a sample for the experiments herein.

### Example 2

### Component Analysis of Unsaponifiable Hemp Seed Extract According to Present Disclosure

Component analysis was performed on the unsaponifiable hemp seed extract of the present disclosure obtained in <Example 1>. The component analysis for the active ingredient was performed by high-performance liquid chromatography (HPLC) and gas chromatography (GC).

Specifically, tocopherol and tocotrienol were analyzed by high-performance liquid chromatography (HPLC) a equipped with LiChrosphere® Diol 100 column (with an inside diameter of 250×4 mm, 5 mm, purchased from Merck, Darmstadt, Germany). A hexane/isopropanol (at a 98.9:1.1 volume ratio) solvent was used as the mobile phase, and the flow rate was set to 1.0 mL/min. The concentrations of tocopherol and tocotrienol were confirmed using a fluorescence detector at excitation and emission wavelengths of 290 nm and 320 nm, respectively. For quantitative analysis, linearity and correlation (R2) were confirmed by obtaining a calibration equation for the peak area at the concentration of each standard substance.

Additionally, carotenoids were analyzed by HPLC (JASCO UV 2075) equipped with a UV/Visible detector, and carotenoids were isolated using Vydac 201TP, C18 Vydac 201TP, C18 (260 mm×4.6 mm, 5 μm, purchased from The Separations Group) as an analysis column. The column temperature was maintained at 30° C., and a solvent in which acetonitrile and methanol (acetonitrile:methanol=7:3, v/v) were mixed was used as the mobile phase. The wavelength of the UV/Visible detector was set to an excitation wavelength of 450 nm, the flow rate was 0.6 mL/min, and the injection volume of the sample per time was 20 μL. Identification was performed by comparing the retention time to those of the standard substances of β-carotene and lutein. For quantitative analysis, linearity and correlation (R2) were confirmed by obtaining a calibration equation for the peak area at the concentration of each standard substance.

Additionally, the concentrations of policosanol and phytosterols were analyzed by GC using 5α-cholestane as an internal standard, where the GC (Varian 3800 purchased from Varian Inc, Walnut Creek, CA, USA) was equipped with a SAC-5 fused-silica capillary column (30 m, with an inner diameter of 0.25 mm, purchased from Supelco, Bellefonte, PA, USA) and a flame ionization detector (FID). The column temperature was set to be maintained at a temperature of 280° C. for 1 minute, raised to 300° C. at a rate of 2.0° C./min, and then maintained at 300° C. for 20 minutes. Helium was used as the mobile phase gas, and the total gas flow rate was 20 mL/min. The temperatures of the sample injector and the detector were set to 310° C. and 320° C., respectively. Policosanol and phytosterols were analyzed by comparing the retention time to those of the standard substances.

The analysis results of the active ingredient of the unsaponifiable hemp seed extract, analyzed by HPLC and GC, are shown in Table 1 below.

TABLE 1
Phytochemical contents of unsaponifiable
matter from hemp seed (HUSM)

| Phytochemicals | | (mg/100 g of HSUM) |
|---|---|---|
| Carotenoids | Lutein | 44.67 ± 0.73 |
| | Zeazanthin | — |
| | α-Carotene | — |
| | β-Carotene | 3.83 ± 0.03 |
| | Total | 48.50 ± 0.03 |
| Vitamin E | α-Tocopherol | 668.75 ± 0.14 |
| | β-Tocopherol | 27.52 ± 4.54 |
| | γ-Tocopherol | 9,058.79 ± 0.24 |
| | δ-Tocopherol | 202.31 ± 1.75 |
| | α-Tocotrienol | — |
| | β-Tocotrienol | — |
| | γ-Tocotrienol | 14.85 ± 4.72 |
| | δ-Tocotrienol | — |
| | Total | 10,074.62 ± 168.84 |
| Phytosterol | Campesterol | 2,517.88 ± 0.28 |
| | Stigmasterol | 361.50 ± 0.35 |
| | β-Sitosterol | 108.04 ± 0.12 |
| | Total | 13,683.50 ± 48.81 |
| Policosanol | C22 | 49.98 ± 5.99 |
| | C24 | 138.97 ± 0.88 |
| | Total | 187.95 ± 5.88 |

As shown in Table 1, it was confirmed through the results that the unsaponifiable hemp seed extract of the present disclosure contained high contents of vitamin E derivatives (α-tocopherol, β-tocopherol, γ-tocopherol, and γ-tocotrienol), known to prevent oxidative damage in the human body, and policosanol (C22 and C24), known to reduce cholesterol levels in the body. Additionally, phytosterols (campesterol, stigmasterol, and β-sitosterol), known as plant sterols, and carotenoids (lutein and β-carotene), which are red-yellow pigments, were also detected. In particular, it was confirmed that the unsaponifiable hemp seed extract had a vitamin E content of about 9.95% and a phytosterol content of about 13.68%. Additionally, the high contents of the vitamin E and phytosterols, contained in the extract, were expected to reduce blood cholesterol levels and low-density lipoprotein (LDL) cholesterol levels. As a result, experiments in the following examples were performed using the hemp seed extract.

### Example 3

Confirmation of Lipogenesis Inhibitory Efficacy by Treatment with Unsaponifiable Hemp Seed Extract of Present Disclosure in Adipocytes

The inventors of the present disclosure analyzed lipogenesis inhibitory activities by treatment of adipocytes with the extract to confirm whether the unsaponifiable hemp seed extract of the present disclosure had anti-obesity activities. To this end, 3T3-L1 preadipocytes were used as adipocytes, purchased from ATCC (CL-193, Manassas, VA, USA), a DMEM medium containing 10% BCS was used for cell proliferation, and 10% FBS DMEM was used as a medium for differentiation induction and maturation. The preadipocytes were dispensed onto a culture plate and then induced to be differentiated into adipocytes for two days using 10% FBS DMEM supplemented with 5 μg/mL insulin, 0.5 mM IBMX, and 1 μM Dex once the cells reached the confluent stage. In this case, the extract of the present disclosure was added to the medium at varying concentrations ranging from 0 to 200 μg/mL. After the induction of differentiation was completed, the adipocytes were matured for 4 days using a medium containing only insulin. Next, the medium was removed after the preadipocytes were induced to be differentiated into adipocytes. Then, the resulting cells were washed several times with PBS, fixed using a 108 formalin solution, and stained using a 0.3% Oil Red O solution capable of specifically reacting with lipid droplets produced in the cells. The stained cells were observed with a microscope and dissolved using isopropanol. Subsequently, the absorbance was measured at 450 nm, and the level of lipogenesis was analyzed as a percentage of the absorbance value relative to that of the control group.

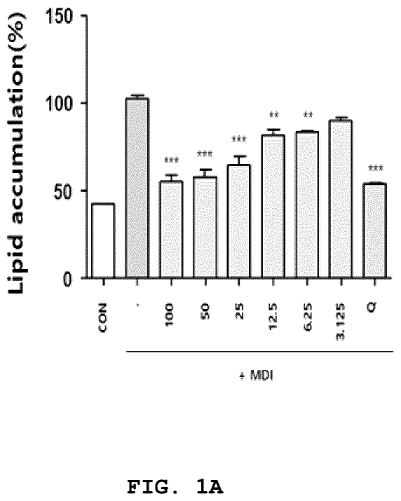

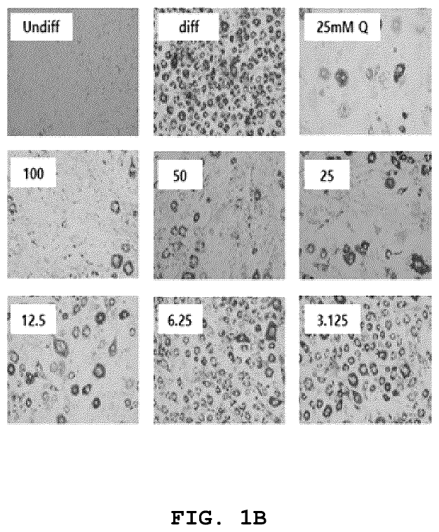

As shown in FIGS. 1A and 1B, it was seen from the results that the number of adipocytes produced with adipocyte differentiation and cell maturation significantly increased in the case of the group where the 3T3-L1 preadipocytes were treated with a differentiation inducer (MDI), compared to that in the case of the group where the 3T3-L1 preadipocytes were not treated with the differentiation inducer. On the contrary, in the case of the group treated with the unsaponifiable hemp seed extract of the present disclosure, it was shown that triglyceride accumulation in the adipocytes was inhibited in a treatment concentration-dependent manner. Additionally, in the case of the group treated with the extract of the present disclosure at concentrations of 100 and 50 μg/mL, it was shown that triglyceride accumulation was inhibited to a similar level as in the case of the group treated with quercetin (25 mM), known to have the ability to inhibit adipocyte differentiation.

Through these results, the inventors of the present disclosure found that the unsaponifiable hemp seed extract of the present disclosure had a lipogenesis inhibitory efficacy.

### Example 4

Confirmation of Anti-Obesity Efficacy in Obesity-Induced Mouse Model by Treatment with Unsaponifiable Hemp Seed Extract of Present Disclosure

The inventors of the present disclosure performed analysis on a mouse animal model in which obesity was induced by a high-fat diet to confirm whether the unsaponifiable hemp seed extract of the present disclosure was actually able to induce anti-obesity effects.

To this end, 4-week-old C57BL/6J mice as experimental animals were bred for a domestication and breeding period of 7 days under the following conditions: a temperature of 21±2° C., a relative humidity of 50±20%, a ventilation frequency of 10 times/hr, a lighting time of 12 hours (lights on at 8 a.m. and lights off at 8 p.m.), and an illuminance of 150 to 300 Lux. Six animals were each independently placed in four experimental groups: a normal diet group (ND), a high-fat diet group (HFD), a high-fat diet group to which the unsaponifiable hemp seed extract was orally administered at 50 mg/kg/day (HFD-HSUM 50), and a high-fat diet group to which the unsaponifiable hemp seed extract was orally administered at 100 mg/kg/day (HFD-HSUM 100). Then, the body weight gain and food intake of each group were compared. The normal diet group was provided with a 10 kcal % fat diet (D12450B, purchased from Research Diets, Inc., New Brunswick, NJ, USA), and the high-fat diet groups were provided with a 60 kcal % high-fat diet (D12451, purchased from Research Diets, Inc., New Brunswick, NJ, USA) on sale. During the breeding period of the experimental animals, the food intake was measured at regular times every day, and the body weight was measured once a week. The food efficiency ratio (FER) was calculated as a ratio of the food intake (g/day) relative to the body weight gain (g/day).

Additionally, after the mice in each experimental group were killed, the adipose tissue, liver, and blood were isolated from each mouse. Then, the adipose tissue and liver were weighed and subjected to H&E staining to perform histopathological observation with an optical microscope. The levels of total cholesterol, LDL cholesterol, total neutral lipid (TG), aspartate aminotransferase (AST), and alanine aminotransferase (ALT) in the blood plasma were analyzed using kits on sale.

### <4-1> Analysis of Body Weight Change and Food Efficiency Ratio

The body weight of each mouse in the experimental groups was measured for 60 days according to the experimental method described above, and FER was analyzed.

TABLE 2
Effect of HSUM on body weight gain, food
intake, and FER in HFD-induced obese mice

| | | | Body | | |
|---|---|---|---|---|---|
| | Body weight (g) | | weight gain | Food intake | |
| Group | Initial | Final | (g/8 weeks) | (g/day) | FER (%) |
| ND | 18.27 ± 0.41 | 24.60 ± 1.13^{c} | 6.38 ± 1.18^{c} | 2.61 ± 0.10^{a} | 0.35 ± 0.02^{b} |
| HFD | 18.95 ± 1.25 | 31.44 ± 1.89^{a} | 12.15 ± 0.99^{a} | 2.39 ± 0.16^{b} | 0.63 ± 0.03^{a} |
| HSUM 50 | 18.90 ± 0.92 | 28.25 ± 1.21^{b} | 9.39 ± 1.27^{b} | 2.15 ± 0.08^{b} | 0.62 ± 0.02^{a} |
| HSUM 100 | 19.15 ± 0.74 | 28.25 ± 1.39^{b} | 9.25 ± 1.76^{b} | 2.13 ± 0.08^{b} | 0.66 ± 0.09^{a} |

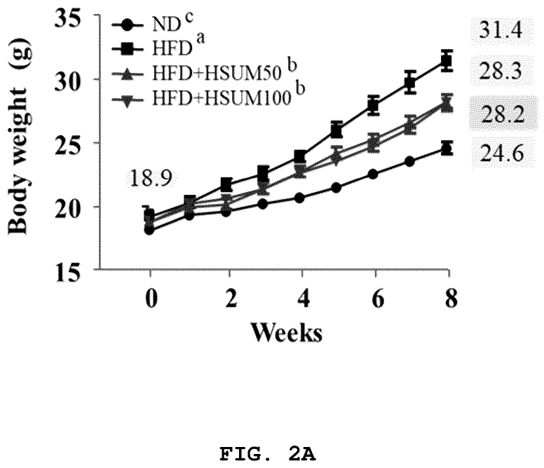

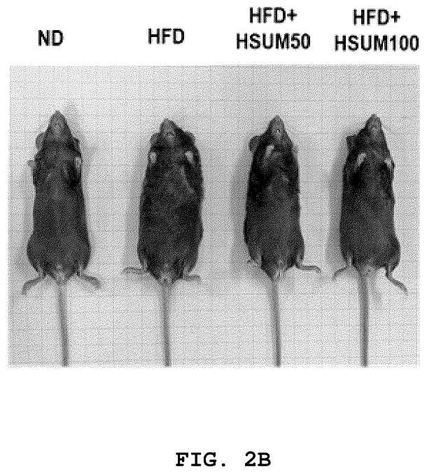

As shown in Table 2 and FIGS. 2A and 2B, although the initial body weight did not show significant differences in all the experimental groups, it was seen from the results that after 8 weeks of food intake, the body weight of mice in the high-fat diet (HFD) group significantly increased compared to that in the normal diet, indicating that obesity was induced. Compared to the high-fat diet (HFD) group, the groups treated with the unsaponifiable hemp seed extract of the present disclosure showed that the body weight effectively decreased, confirming that the unsaponifiable hemp seed extract of the present disclosure had an anti-obesity efficacy.

### <4-2> Measurement of Changes in Weight of Liver and Adipose Tissue

The liver and fat were isolated from each mouse used in the experiment according to the experimental method described above, and the weight thereof was measured.

TABLE 3
Effect of HSUM on liver and adipose tissue
weights in HFD-induced obese mice

| | | Epididymal white | Mesenteric white |
|---|---|---|---|
| Group | Liver | adipose tissue | adipose tissue |
| ND | 1.00 ± 0.06^{a} | 0.59 ± 0.07^{c} | 0.46 ± 0.02^{b} |
| HFD | 0.95 ± 0.04^{ab} | 1.83 ± 0.09^{a} | 0.70 ± 0.05^{a} |
| HSUM 50 | 0.84 ± 0.02^{b} | 1.27 ± 0.11^{b} | 0.41 ± 0.03^{b} |
| HSUM 100 | 0.81 ± 0.03^{b} | 1.31 ± 0.11^{b} | 0.49 ± 0.04^{b} |

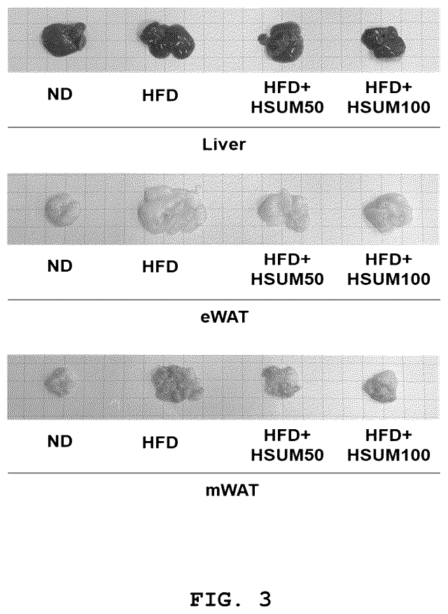

As shown in Table 3 and FIG. 3, it was seen from the results that the weight of epididymal fat and mesenteric fat in the case of the group to which the unsaponifiable hemp seed extract of the present disclosure was administered significantly decreased compared to that in the case of the high-fat diet group.

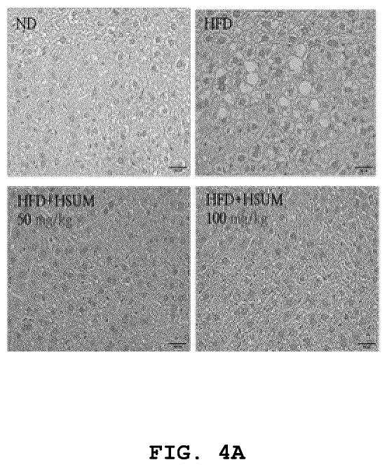

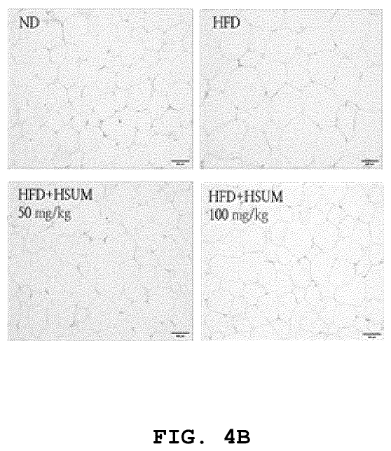

Additionally, as shown in FIGS. 4A and 4B, it was seen from the results of the liver and adipose tissue of each mouse, pathologically observed with an optical microscope, through H&E staining experiments that lipid droplets in the liver tissue appeared extensively in the case of the high-fat diet group compared to those in the case of the normal diet group. On the other hand, in the case of all the groups to which the unsaponifiable hemp seed extract of the present disclosure was administered, the number of lipid droplets was confirmed to be reduced. Additionally, while such results were observed similarly in epididymal adipose tissue, the adipocyte size appeared to increase in the case of the high-fat diet group compared to those in the case of the normal diet group. On the other hand, in the case of the group to which the unsaponifiable hemp seed extract of the present disclosure was administered, the adipocyte size was confirmed to be greatly reduced.

### <4-3> Analysis of AST and ALT Activities and Changes in Concentrations of Plasma Lipid and Cholesterol

Furthermore, the inventors of the present disclosure analyzed AST and ALT activities to confirm whether the weight loss in <4-1> above resulted from changes associated with liver toxicity.

As a result, the AST and ALT activities did not exhibit significant differences in all the experimental groups and thus were proven to have no relationship with liver toxicity.

TABLE 4
Effect of HUSM on liver and adipose tissue weights in HFD-induced obese mice

| Group | ND | HFD | HSUM 50 | HSUM 100 |
|---|---|---|---|---|
| Triglyceride (mg/dL) | 39.34 ± 9.53^{b} | 69.37 ± 13.93^{a} | 38.69 ± 6.07^{b} | 38.26 ± 9.01^{b} |
| Total cholesterol (mg/dL) | 83.81 ± 16.92^{c} | 159.58 ± 12.45^{a} | 139.60 ± 12.61^{ab} | 132.01 ± 16.60^{b} |
| LDL cholesterol (mg/dL) | 88.07 ± 14.40^{c} | 167.34 ± 9.50^{a} | 141.50 ± 11.26^{ab} | 134.05 ± 14.73^{b} |
| ALT (U/L)^{NS} | 27.13 ± 17.05 | 19.80 ± 9.57 | 18.08 ± 13.70 | 19.50 ± 10.99 |
| AST (U/L)^{NS} | 2.53 ± 1.28 | 1.84 ± 1.46 | 1.94 ± 3.36 | 2.43 ± 2.60 |

Hence, the inventors of the present disclosure analyzed the total triglyceride, total cholesterol, and LDL cholesterol contents in the blood of each mouse in the experimental groups. As shown in Table 4, it was seen from the results that the contents of the components mentioned above were significantly higher in the case of the high-fat diet groups than those in the case of the normal diet group. However, the contents of these components were significantly reduced in the case of the groups to which the unsaponifiable hemp seed extract of the present disclosure was administered, compared to those in the case of the HFD group.

These results show that the unsaponifiable hemp seed extract of the present disclosure is effectively usable to prevent, reduce, and treat obesity due to not causing liver toxicity and having the efficacy of reducing total triglyceride, total cholesterol, and LDL cholesterol levels in the blood, increased by obesity.

Hereinabove, the present disclosure has been described above in terms of the preferred embodiments. Those skilled in the art to which the present disclosure belongs will appreciate that various modifications and other equivalent embodiments are possible without departing from the essential features of the present disclosure. Accordingly, the disclosed embodiments should be considered from an illustrative, rather than a limiting, perspective. The scope of the present disclosure should be defined by the technical spirit of the claims, not by the foregoing description, and should be construed as covering the spirit of the present disclosure defined by the claims of the present disclosure and all modifications, equivalents, and substitutes falling within the scope of the present disclosure.

What is claimed is:

1. A food composition for preventing or reducing obesity, the composition comprising a hemp seed extract as an active ingredient.

2. The food composition of claim 1, wherein the hemp seed extract is an unsaponifiable hemp seed extract.

3. The food composition of claim 1, wherein the hemp seed extract has the following activities:
inhibiting lipid droplet formation and adipocyte differentiation and reducing an adipocyte size, when inducing preadipocytes to be differentiated into adipocytes, and
reducing total triglyceride, total cholesterol, and low-density lipoprotein (LDL) cholesterol levels.

4. A pharmaceutical composition for preventing or treating obesity, the composition comprising a hemp seed extract as an active ingredient.

5. The pharmaceutical composition of claim 4, wherein the hemp seed extract is an unsaponifiable hemp seed extract.

6. The pharmaceutical composition of claim 4, wherein the hemp seed extract has the following activities:
inhibiting lipid droplet formation and adipocyte differentiation and reducing an adipocyte size, when inducing preadipocytes to be differentiated into adipocytes, and
reducing total triglyceride, total cholesterol, and LDL cholesterol levels.

7. A method of preparing a hemp seed unsaponifiable extract having an anti-obesity activity, the method comprising:
(1) adding a hemp seed, pyrogallol, and potassium hydroxide (KOH) to a reaction vessel and filling the reaction vessel with nitrogen;
(2) causing a reaction in a water bath at a temperature in a range of 70° C. to 80° C. for 40 to 60 minutes and then cooling the resulting product; and
(3) adding sodium chloride (NaCl) and an extraction solvent in which dibutylhydroxytoluene (BHT) hexane and ethyl acetate are mixed to the cooled product and shaking the resulting mixture to obtain a supernatant.
